(19) United States
(12) Patent Application Publication
(10) Pub. No.: US 2023/0248961 A1
(43) Pub. Date: Aug. 10, 2023

(54) CARDIAC ASSIST DEVICE

(71) Applicant: CardiacBooster B.V., Nijmegen (NL)

(72) Inventors: Daniël Immanuel Michaël van Dort, Nijmegen (NL); Florian Niklas Ludwig, Hilversum (NL)

(21) Appl. No.: 18/300,207

(22) Filed: Apr. 13, 2023

Related U.S. Application Data
(63) Continuation of application No. PCT/NL2021/050621, filed on Oct. 14, 2021.

(30) Foreign Application Priority Data
Oct. 14, 2020 (NL) 2026671

(51) Int. Cl. A61M 60/174; A61M 60/295; A61M 60/515

(52) U.S. Cl. CPC A61M 60/174; A61M 60/295; A61M 60/515; A61M 2205/0266

(57) ABSTRACT

A cardiac assist device (1) with a cup element (2), an inner balloon element (5) and a tube element (6). The cup element (2) has a cup wall (2a), one or more in-flow openings (3), and an outflow element (4 having an aperture (4a). The inner balloon element (5) is positioned inside the cup element (2) free from the outflow element (4). The tube element (6) is arranged for inflating and deflating the inner balloon element (5) during operation. During operation in a pumping operational mode, the combination of first material, dimensions of the cup wall (2a), and dimensions of the outflow element (4) provides a containment force by the cup element (2) counteracting an outward directed force of the inner balloon element (5).

### CROSS-REFERENCE TO RELATED APPLICATIONS

The present application is a continuation of International Application No. PCT/NL2021/050621, filed Oct. 14, 2021, which claims the benefit of priority to Dutch Patent Application No. 2026671, filed Oct. 14, 2020, each of which is incorporated by reference herein in its entirety.

### FIELD OF THE INVENTION

The present invention relates to a cardiac assist device comprising a cup element, an inner balloon element and a tube element.

### BACKGROUND ART

U.S. Pat. No. 5,169,378 describes an intraventricular assist pump. The pump comprises a body pump, or external chamber, having a double lumen wall, that is expansible and of variable rigidity, i.e. an inflatable but static outer cup. In operation the (inflatable) external chamber is rigid and static during operation. A transvalvular segment, or flexible neck of the pump, is provided that conforms itself to the situation or position of “open” or “closed” of the aortic or pulmonary valves and avoids the need of using a valve in the discharge of blood from the pump. An internal balloon having a progressive wall thickness is provided and causes a sequential rhythm of inflation and deflation.

International patent publication WO2015/131879 discloses a catheter device for conducting a fluid, in particular a bodily fluid, in a directed manner. The catheter device is described as being positioned in the aorta, and comprises a shell having an interior and comprising a frame, wherein the shell comprises at least three openings and is designed as a line for the fluid in a region between a first opening and a second opening, and wherein a non-return valve is arranged at the second opening. During operation, the shell is in an expanded state and fully rigid (e.g. implemented as a stent). The non-return valve comprises a valve film, which is at least partially fastened to the shell such that the second opening can be completely covered by the valve film.

International patent application WO2020/022905 discloses a heart support device for circulatory assistance having a chamber body with a first opening. A dynamic volume body is provided for increasing and decreasing the interior volume of the chamber body. The outflow of blood during operation is directed using a directional flow structure.

### SUMMARY OF THE INVENTION

The present invention seeks to provide an improved cardiac assist device allowing proper and efficient operation.

According to the present invention, a cardiac assist device as defined above is provided, in which the cardiac assist device comprises a cup element having a cup wall comprising a first material and defining an inner cup volume, one or more in-flow openings arranged in the cup wall to allow a first fluid (such as blood) to flow into the cup element during operation, and an outflow element connected in fluid communication with the cup wall and having an aperture for expelling the first fluid during operation. An inner balloon element is present having a balloon wall comprising a second material and defining an inner balloon volume, the inner balloon element being positioned inside the cup element free from the outflow element. A tube element is provided in fluid communication with the inner balloon element for inflating and deflating the inner balloon element during operation, creating a pumping operational mode and a filling operational mode, respectively. During operation in the pumping operational mode, the combination of first material, dimensions of the cup wall, and dimensions of the outflow element provides a containment force by the cup element counteracting an outward directed force of the inner balloon element.

The present invention embodiments allow a very good and efficient operation of the cardiac assist device with a very limited number of components.

### BRIEF DESCRIPTION OF THE DRAWINGS

The present invention will be discussed in more detail below, with reference to the attached drawings, in which invention;

FIGS. 1A and 1B show cross sectional views of a first embodiment of the present

FIG. 2A-2D show cross sectional views of the second embodiment of the present invention;

FIG. 3A shows a perspective view of a third embodiment of the present invention, and FIG. 3B shows a cross sectional view of an alternative embodiment of the skeleton structure for the embodiment shown in FIG. 3A;

FIG. 4 shows a side view of a further embodiment of the cardiac assist device according to the present invention,

FIG. 5 shows a cross sectional view along the line V-V in FIG. 4,

FIG. 6 shows a cross sectional view along the line VI-VI in FIG. 4,

FIGS. 7A and 7B show a cross sectional view along the line VII-VII in FIG. 4 in two operational states,

FIG. 8A-C show perspective views of (parts of) yet a further embodiment of the present invention,

FIG. 9 shows an exploded perspective view of one-way valves used in a further embodiment of the present invention,

FIG. 10 shows a side view of an even further embodiment of the cardiac assist device according to the present invention,

FIG. 11 shows a perspective view of an embodiment of the cardiac assist device with details on retrieve elements, and

FIG. 12 shows a cross sectional view of an embodiment of the present invention cardiac assist device.

### DESCRIPTION OF EMBODIMENTS

The present invention seeks to provide an intra-lumen cardiac assist device that more closely approximates the natural function of the heart. The resulting cardiac assist device according to the present invention embodiments can function to provide circulatory assistance in a patient by pumping blood from a cardiovascular lumen (e.g. the left ventricular chamber) with a sufficiently high stroke volume and efficient placement.

In the present invention embodiments, two pumping mechanisms are implemented that cooperate and make the functioning of the cardiac assist device more efficient.

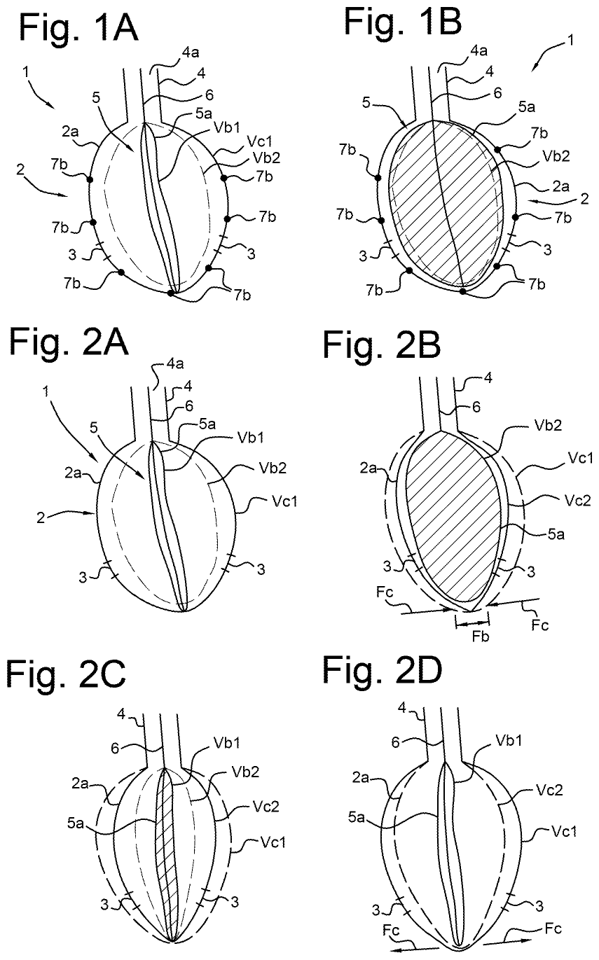

FIGS. 1A and 1 B show two cross sectional views of a first embodiment of the present invention cardiac assist device 1, FIG. 1A showing the cardiac assist device 1 at the start of a pumping operational mode, and FIG. 1 B showing the cardiac assist device 1 at the start of a filling operational mode, wherein the two operational modes are alternating. The cardiac assist device 1 e.g. has a generally ellipsoid or ovoid outer shape, with a longitudinal axis of symmetry.

The cardiac assist device 1 comprises a cup element 2 and an inner balloon element 5, the inner balloon element 5 inflating during the pumping operational mode and deflating during the filling operational mode. The inner balloon element 5 has a balloon wall Sa, and the cup element 2 has a cup wall 2a, which is provided with inflow openings 3, allowing a first fluid (blood) to enter the space between cup wall 2a and balloon wall Sa. The cup element 2 further comprises an outflow element 4 connected in fluid communication with the cup wall 2a and having an aperture 4a for expelling the first fluid during operation. A tube element 6 is present in fluid communication with the inner balloon element 5, and serves for inflating and deflating the inner balloon element 5 during operation, creating a pumping operational mode and a filling operational mode, respectively. As shown in FIG. 1A, the cup element 2 has an inner cup volume Vc1, which is substantially the same during the pumping operational mode and the filling operational mode. The inner balloon element has an initial inner volume Vb1 at start of pumping operational mode as shown in FIG. 1A and an expanded inner volume Vb2 at the start of the filling operational mode as shown in FIG. 1B. The obtainable stroke volume SV of the cardiac assist device 1 of this embodiment is thus Vb2−Vb1.

To obtain this pumping and filling operational modes, the one or more inflow openings 3 need to be closed off during the pumping operational mode, and open during the filling operational mode. This may be achieved as described below, or by a further group of embodiments, wherein the one or more inflow openings 3 comprise one-way valves.

Furthermore, in a group of embodiments, the outflow element 4 has a tube like structure. The tube like structure may be dimensioned long enough to extend through the aortic valve when 30 the cardiac assist device 1 is positioned in the left ventricle (e.g. having a length of at least 20 mm), ensuring the aperture 4a is in the aorta during operation. Additionally or alternatively, the outflow element 4 is a directional flow element. This allows to obtain a directed flow of first fluid during the pumping operational mode, e.g. directed towards the aortic valve during operation.

The cardiac assist device 1 of the present invention, in fact combines two pumping mechanisms in order to obtain a small as possible outer volume of the cardiac assist device 1 with a high as possible stroke volume, with an unobstructed as possible outflow of first fluid through the outflow element 4, This is made possible by the (dynamic) balance of forces during the alternating pumping operational mode and filling operational mode.

More specifically, during operation in the pumping operational mode, the combination of a first material of the cup wall 2a, dimensions of the cup wall 2a, and dimensions of the outflow element 4 provides a containment force by the cup element 2 counteracting an outward directed force of the inner balloon element 5 being inflated. These structural features of the cup wall 2a (first material properties, dimensions of cup wall 2a (thickness, radius, surface area), and dimensions of the outflow element 4 (area of opening 4a, diameter and/or length of outflow element 4) determine 5 the containment force. It is noted that further parameters may be relevant during operation, such as the speed in volume change of the stroke volume, the resistance over the outflow element 4, the viscosity of the first fluid, and/or back pressure from the outside environment of the cardiac assist device, (such as the aortic pressure in case of ventricular assist type of use of the cardiac assist device 1). However, these can be taken into account when setting the structural features of the cup element 2.

Furthermore, during the filling operational mode, the structure and material of the cup element 2 ensures that the shape of the cup wall 2a remains substantially the same (i.e. with inner cup volume Vc1), counteracting a force generated by the deflating inner balloon element 5. It is noted that further parameters in this case may be relevant during operation, such as the resistance over the total inflow surface area of the inflow openings 3 in the cup wall 2a, the speed of deflation of the inner balloon element 5, and the viscosity of the first fluid. Again, these further parameters can be taken into account when selecting the structural features of the cup element 2 for the overall design of the cardiac assist device 1.

Thus, in general wording, the present invention provides a cardiac assist device 1 comprising a cup element 2 having a cup wall 2a comprising a first material and defining an inner cup volume Vc1, one or more in-flow openings 3 arranged in the cup wall 2a to allow a first fluid to flow into the cup element 2 during operation, an outflow element 4 connected in fluid communication with the cup wall 2a and having an aperture 4a for expelling the first fluid during operation. An inner balloon element 5 is present having a balloon wall Sa comprising a second material and defining an inner balloon volume Vb1; Vb2, the inner balloon element 5 being positioned inside the cup element 2 free from the outflow element 4, and a tube element 6 in fluid communication with the inner balloon element 5 for inflating and deflating the inner balloon element 5 during operation, creating a pumping operational mode and a filling operational mode, respectively. During operation in the pumping operational mode, the combination of first material, dimensions of the cup wall 2a, and dimensions of the outflow element 4 provides a containment force by the cup element 2 counteracting an outward directed force of the inner balloon element 5.

In a group of embodiments, during the pumping and filling operational modes, the cup element 2 has a substantially constant inner cup volume Vc1.

In the exemplary embodiment shown in FIGS. 1A and 1 B, furthermore, the cup element 2 is provided with a skeleton (or reinforcement) structure 7b, e.g. as an integral part of the cup wall 2a, providing rigidity to the cup wall 2a. In other words, in further embodiments, the cup element 2 comprises a skeleton structure 7a, 7b integrated with the cup wall 2a.

In a further group of embodiments, the cup element 2 has a dynamic inner cup volume Vc1-Vc2 as shown in the exemplary embodiment shown in cross section in FIG. 2A-2D. The cup wall 2a will co-operate synchronously with the inflating/deflating balloon element 5 during operation, to obtain an even more effective stroke volume SV. In sequence, FIG. 2a-2D show there are two major actions in the cardiac assist device 1, i.e. in FIG. 2A the cup element 2 is kept fully open (i.e. maximum inner cup volume Vc1) until the inner balloon element 5 is fully deflated and the inner volume is filled with the first fluid via the one or more in-flow openings 3. FIG. 2B then shows the next step in the sequence, wherein the cup wall 2a is contracted (or tightened) to a minimum inner cup volume Vc2, and the inner balloon element 5 is inflated from inner balloon volume Vb1 to Vb2, thereby combining the two forces Fe and Fb for the pumping operational mode. Subsequently, the inner balloon element 5 is allowed to deflate again, filling the inner volume of the cardiac assist device 1 again with the first fluid (filling operational mode), which is continued with an increase of the inner cup volume to its maximum level Vc1 again (FIG. 2D). Then a new cycle can start again.

In one group of embodiments, the varying dynamic volume of the cup element 2 is achieved by proper selection of structural features of the cup element 2, such as choice of first material, dimensions of the cup wall 2a, and dimensions of the outflow element 4.

In a further group of embodiments, this dynamic volume of the cup element 2 is obtained by having a skeleton structure 7a, 7b which is a flexible skeleton structure arranged to control the cup volume during operation. Note that the skeleton structure 7b shown in the exemplary embodiment of FIGS. 1A and 1B can also be applied to the exemplary embodiment shown in FIG. 2A-2D.

In a further embodiment, the flexible skeleton structure 7a, 7b comprises hollow channels, which can e.g. be inflated and deflated to obtain the varying inner cup volume Vc1-Vc2. To that end, the hollow channels are in fluid communication with the tube element 6 in a further embodiment. Alternatively, the cardiac assist device 1 further comprises a secondary tube element in communication with the hollow channels. All these components allow operation of the cardiac assist device 1 according to these embodiments with a relatively high pressure, e.g. between 0.5 and 20 bar. As this is higher than prior art systems which operate with an inflation pressure of 0.5-1 bar (see e.g. US patent publication U.S. Pat. No. 5,169,378), a quicker and more robust operation of the cardiac assist device 1 is possible.

In order to obtain the containment force counteracting the inflating force of the inner balloon, the skeleton structure 7a, 7b comprises shape-memory material in a further group of embodiments. The shape memory material is e.g. wire shaped, and may comprise nitinol as memory material. E.g. the skeleton structure 7b may then be implemented as a helical wire included in the cup wall 2a, as shown in the embodiment of FIGS. 1A and 1B.

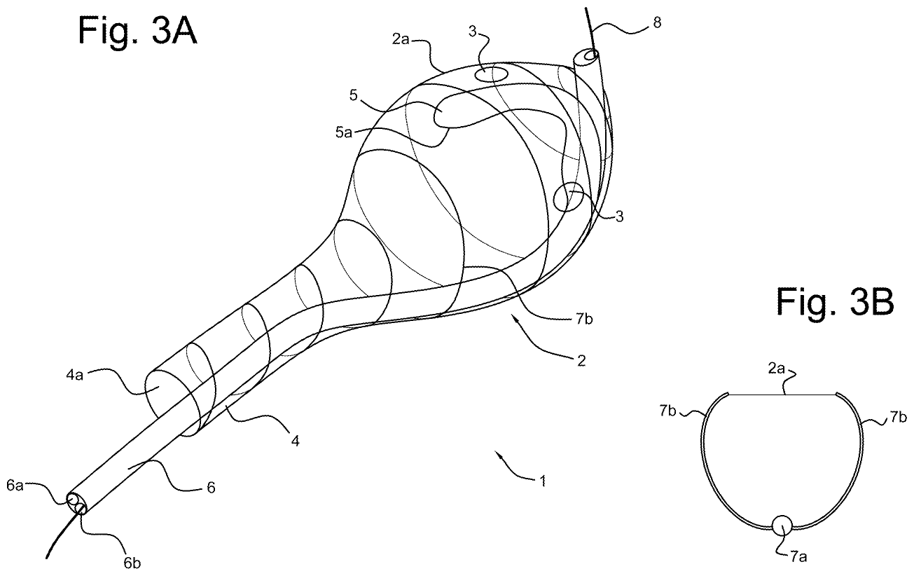

FIG. 3A shows a perspective view of yet a further embodiment of the present invention cardiac assist device 1. In this embodiment, the skeleton structure 7b comprises a helical shaped wire element integrated with the cup wall 2a. FIG. 3B shows a cross sectional view of a further embodiment, wherein the skeleton structure 7a, 7b, comprises a spine element 7a arranged along a longitudinal direction of the cardiac assist device 1 and a plurality of rib elements 7b, each of the plurality of rib elements 7b being attached to the spine element 7a on one side thereof. The rib elements 7b are e.g. extending from the spine element 7a in a substantially perpendicular manner, forming a rib-cage type of skeleton structure. An added benefit of this embodiment is that the rib element 7b can easily fold, allowing more easy entry and exit of the cardiac assist device before being put into operation (e.g. via a vascular catheter into the left ventricle). In an even further alternative embodiment, the spine element 7a and/or rib element 7b may be partially of solid material (e.g. nitinol) and partially open (e.g. hollow polyurethane material).

In an even further group of embodiments, the cardiac assist device further comprises a control unit 10 arranged to control fluid flow of a second fluid through the tube element 6 during operation. This allows to inflate/deflate the inner balloon element 5 periodically to obtain the pumping and filling operational modes as described above. The second fluid can be (compressed) air, a gas, a liquid, water, etc. The tube element 6 is e.g. implemented as a catheter (able to transport the second fluid), allowing the use of a remote control of the inflation/deflation of the inner balloon element 5 by a remote pressure source. If present, the hollow channels of the skeleton structure 7a, 7b described above can also be controlled in this manner. E.g. when using a high pressure fluid, the hollow channels will eventually become rigid providing shape consistency of the cup element 2. By adding resistor elements and selecting the inner volumes of hollow channels versus inner balloon 5 dimensions, the same second fluid source and control unit 10 may be used to first inflate the skeleton structure 7a, 7b and subsequently the inner balloon element 5.

In an even further embodiment, the cup element 2 is provided with an inflatable skeleton structure 7a, 7b, wherein the internal volume of the inflatable skeleton 7a, 7b is smaller than the (possible) internal volume of the inner balloon element 5, e.g. 1 cc versus 20 cc. This allows the inflatable skeleton 7a, 7b and the inner balloon element 5 to be connected to a single (remote) pressure source via the tube element 6, as the lower volume will ensure an inflation of the skeleton structure first, and subsequently inflation of the inner balloon element 5. In order to implement the operational use of the present invention embodiment with a dynamic inner cup volume, in a further embodiment the control unit 10 is arranged to control the inner cup volume Vc1; Vc2 and the inner 25 balloon volume Vb1; Vb2 independently.

The control unit 10 may be arranged to apply a specific synchronization timing between inflating/deflating the cup element 2 and inflating/deflating the inner balloon element 5. The cup element 2 may change from inner cup volume Vc1 to Vc2 just before, simultaneously with or just after the change of inner balloon volume form Vb1 to Vb2. In a specific embodiment, the maximum inner cup volume Vc1 may be timed Oust) prior to the inflation of the inner balloon element 5, thus ensuring an optimal stroke volume.

Furthermore, in an even further exemplary embodiment, the frequency of pumping may be set by the control unit 10 to obtain an optimal performance. This frequency of pumping may be controlled synchronous to an actual (sensed) heartbeat. Even further, the frequency of pumping may be higher, e.g. a factor of 2-10 higher than the actual heartbeat, to obtain a higher flow of the first fluid.

As shown in the exemplary embodiment of FIG. 3, the tube element 6 is provided in contact with the outflow element 4 along a predetermined length thereof in a further embodiment. This off-centre positioning of the tube element 6 allows a generally unobstructed flow of the first fluid in the outflow element 4, and out of the aperture 4a.

The combination of the cup element 2 and the inner balloon element 5 are adjustable in a further embodiment to a transport mode, in which the maximum diameter of the combination is less than 7 mm, e.g. less than 5 mm. This e.g. allows to bring the cardiac assist device to the left ventricle via the aorta and a regular catheter system in a reliable and safe manner. The combination is e.g. elongated in the transport mode, and ellipsoid shaped in an operational mode.

In the exemplary embodiment shown in FIG. 3A, the tube element 6 is a multi-lumen (e.g. dual lumen) catheter. One of the lumens 6a is e.g. used for inflation/deflation of the inner balloon element 5, and a further lumen 6b is providing space for a guide wire 8, all along the cardiac assist device 1, or even extending therefrom, e.g. for proper positioning of the cardiac assist device 1 in the left ventricle.

As a further alternative embodiment, the inner balloon element 5 comprises a multi-stage balloon assembly having at least two balloon parts with different rigidity material. The multi-stage balloon assembly is e.g. a shaped balloon, or with a controlled volume expansion (or directional thrust). Even further, the shaped balloon 5 may be positioned with respect to the outflow element 4 such that a directional inflation occurs, from the apex of the cup element 2 towards the outflow element 4. Alternatively, or additionally, the shaped balloon 5 may be dimensioned and positioned to close off the in-flow openings 3 at an early stage of inflation, thereby creating a one-way valve assembly. In other words, one of the two balloon parts is positioned to close off the one or more inflow openings 3 in the pumping operational mode.

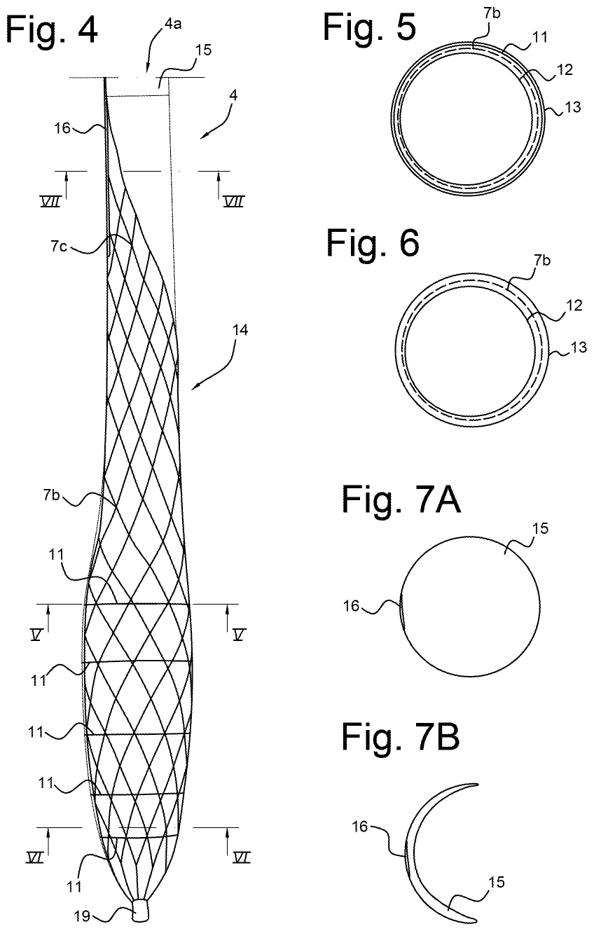

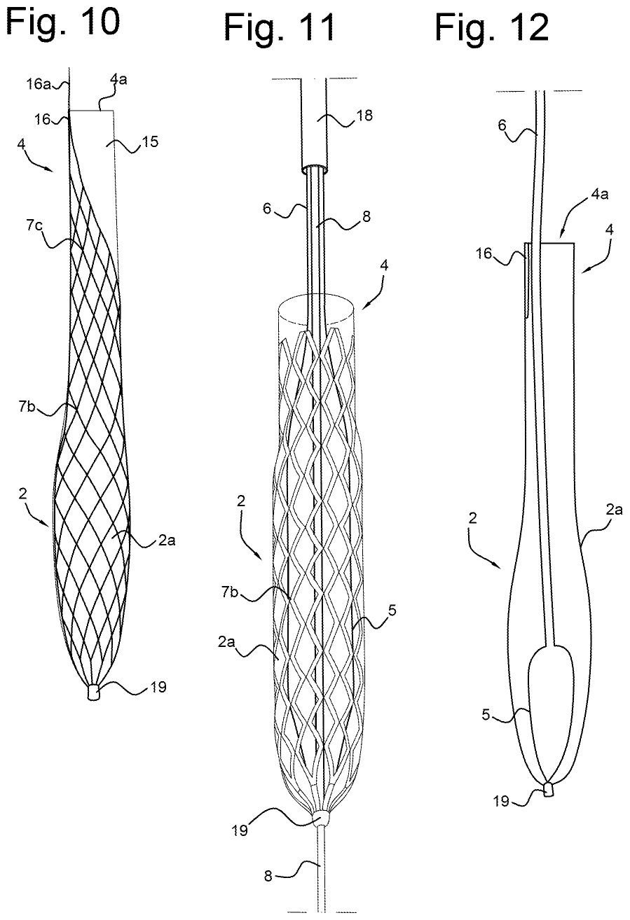

FIG. 4 shows a side view of yet a further embodiment of the cardiac assist device 1 according to the present invention having the cup element 2 and outflow element 4, similar to the embodiments described above. In this exemplary embodiment, the skeleton structure 7b is provided as a wire mesh structure, e.g. using nitinol or another shape-memory material. As an example, the skeleton structure 7b is manufactured similar to a laser cut stent. The skeleton structure 7b in this embodiment allows to decrease the diameter of the cardiac assist device 1 when it is stretched along its longitudinal axis (see also description with reference to FIGS. 10 and 11). Once positioned in the heart, the shape of the cup element 2 returns to its intended shape, with the wire mesh structure ensuring a shape of the cup element 2 having the inner volume Vc1. During operation (both inflation and deflation) the skeleton structure 7b of this embodiment provides a radial stiffness maintaining the inner volume Vc1. At the end of the cup element 2, the skeleton structure 7b and further local components of the cardiac assist device 1 are held together and protected by an end cap 19, which is e.g. shaped or rounded off to provide as little as possible damage to (heart) tissue during use of the cardiac assist device 1.

In order to prevent overstretching of the cup element 2 outer surface during operation, the skeleton structure 7b has a wire mesh structure, provided with one or more circumferential restraining elements 11. The restraining elements 11 are e.g. made of a non-compliant material, ensuring a local maximum diameter of the cup element 2.

FIG. 5 shows a cross sectional view along the line V-V in FIG. 4, and FIG. 6 shows a cross sectional view along the line VI-VI in FIG. 4. In a group of embodiments, the cup wall 2a comprises 40 an inner layer 12 and/or an outer layer 13. The inner and/or outer layer 12, 13 are made of compliant, high stretch force material, or may even be made of non-compliant material (or a combination of compliant and non-compliant material). Furthermore the outer layer 13, spanning across the wire mesh shaped skeleton structure 7b (and the restraining elements 11), also provides a (more) smooth outer surface, such that the cardiac assist device 1 interferes as little as possible with heart tissue (during insertion and during operation).

In the exemplary embodiment shown in FIG. 4-6 the cup element 2 further comprises a trans-valve section 14 in communication with the outflow element 4. In use, this part (in which the skeleton structure 7b extends) can be positioned at the height of e.g. the aortic valve, providing an always open passageway from the inner volume Vc1 of the cup element 2 to the outflow element 4.

FIGS. 7A and 7B show a cross sectional view along the line VII-VII in FIG. 4 in two operational states, which shows the working of the outflow element 4 comprising a collapsible tubular element 15, The collapsible tubular element 15 is made of a material providing radial stiffness (the collapsible tubular element 15 has a maximum diameter, even when fluid is pumped through it at high pressure and/or velocity), yet sufficient flexibility to allow the collapsible tubular element 15 to collapse in order to act as a one-way valve. The open position of the collapsible tubular element 15 is shown in FIG. 7A, and the closed position in FIG. 7B. The collapsible tubular element 15 e.g. has a length of between 0.5 and 4 cm, e.g. 2 cm, and a diameter of between 5 and 15 mm, e.g. 12 mm. The collapsible tubular element 15 is e.g. made from flexible material such as Tecotane 85.

In the exemplary embodiment shown, the outflow element 4 comprises an outflow skeleton structure 7c which is formed by an extension of the skeleton structure 7b. This provides for a partial support of the outflow element 4, compatible with the intended function of the collapsible tubular element 15, and with the intended retrieve mechanism function. In addition or alternatively, the collapsible tubular element 15 is at least partially fixed to the outflow skeleton structure 7c. In addition, or alternatively, the collapsible tubular element 15 may be provided with a shaped outer end to even further improve the one-way valve function, e.g. by providing angulation of the upper end of the collapsible tubular element 15 (e.g. at 40 degrees to the longitudinal direction of the cardiac assist device 1.

This exemplary embodiment of the cardiac assist device 1 has a stiff part (cup element 2) which during operation is positioned in the ventricle, including a trans-valve section 14 which during operation passes through the aortic valve, and a one-way valve mechanism formed by the collapsible tubular element 15 of the outflow element 4. During systole, the stiff part translates energy provided by the action of the internal balloon element 5, pushing fluid towards the outflow element 4. Already at the end of this stage, the collapsible tubular element 15 starts closing when the ejection is over as the fluid flow decreases, causing the pressure inside the collapsible tubular element 15 to be lower than the surrounding pressure. During diastole, the outflow skeleton structure 7c prevents collapse of the outflow element 4, yet the collapsible tubular element 15 remains closed, thus preventing possible back flow of fluid.

Furthermore, as shown in FIGS. 4, 7A and 7B an attachment element 16 is provided at the upper end of the skeleton structure 7b (or even only at the upper end of outflow skeleton structure 7c), which is effective in use when retrieving the cardiac assist device 1. FIG. 10 shows a side view of parts of an even further embodiment of the cardiac assist device 1 according to the present invention, playing a role in positioning and retrieving the cardiac assist device 1. A wire 16a can be attached to the attachment element 16 of the skeleton structure 7b/outflow skeleton structure 7c. This attachment to the tip of the outflow element 4 allows to put tension on the cup element 2 while retracting, elongating the cup element 2 and decreasing its width.

FIG. 11 shows a perspective view of an embodiment of the cardiac assist device 1 with details on retrieve elements. In this perspective view, also the inner balloon element 5 and tube element 6 are shown, as well as guide wire 8, and an outer shaft 18 (forming part of the catheter used for positioning and retracting the cardiac assist device 1).

The tapered shape of the outflow element 4 (more specifically the outflow skeleton structure 7c) allows the cardiac assist device 1 to be retracted into the outer shaft 18. The tube element 6 can act as fixation of the end cap 19 (also attached to the inner balloon element 5), allowing to put sufficient force to stretch the cardiac assist device 1 in order to reduce the diameter thereof in order to (again) fit into the outer shaft 18.

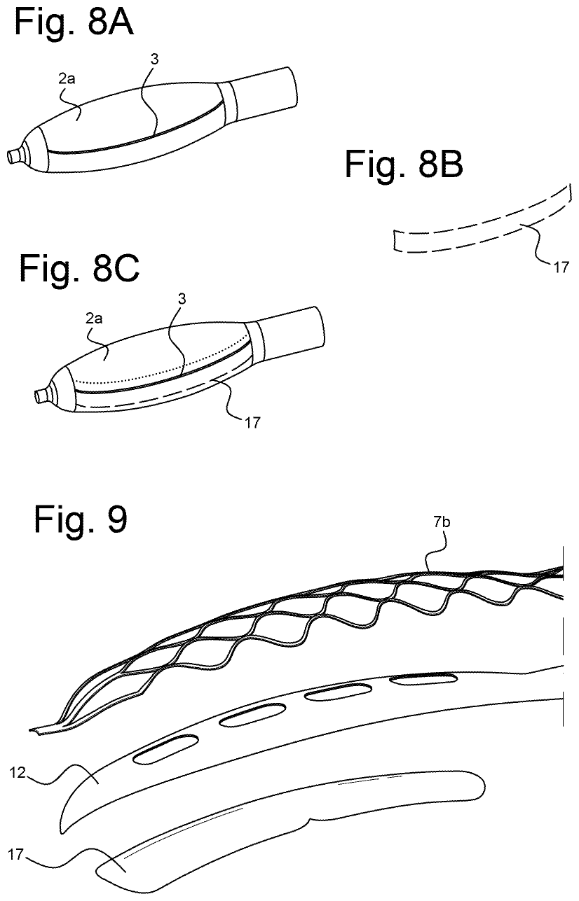

FIG. 8A-C show perspective views of (parts of) yet a further embodiment of the present invention, relating to the in-flow openings 3 of the cardiac assist device 1, and FIG. 9 shows an exploded perspective view of implementations of one-way valves 12, 17 used in a further embodiment of the present invention. In these embodiments, the one or more inflow openings 3 comprise one-way valves 17.

FIG. 8A shows the cup wall 2a of cup element 2, which is provided with a row of inflow openings 3 (rendered as a thick line), and FIG. 8B shows the leaf-like element 17 which in cooperation with the outer surface 2a as acting as one-way inlet valve, In FIG. 8C the mutual positioning is shown, wherein the leaf-like element 17 is attached to the inside surface of cup wall 2a along the dotted line, thereby creating a coaptation area with in the region below the row of inflow openings 3.

FIG. 9 shows an exploded view of a simplified embodiment of the one-way inlet valve implementation. The cup wall 2a is formed by the skeleton structure 7b (partially shown), inner layer 12 (provided with the inflow openings 3) and the leaf-like element 17. The leaf-like element 17 is positioned to allow closure of the inflow openings 3. The coaptation area thus formed e.g. has a dimension of between 0.5 and 4 mm, e.g. 2.5 mm, to allow for a sufficiently rapid opening and closing of the formed one-way inlet valves. The inflow openings 3 are at least 2 mm in diameter, e.g. 3.5 mm. The coaptation area also provides for a directional inflow of fluid inside the cup element 2, with the spiral flow aiding in closing off the one-way inflow valve in a sufficiently quick manner. Multiple such one-way inflow valve lines may be provided in parallel along the longitudinal direction of the cup wall 2a, with the added benefit of an increased directional and spiral flow inside the cup element 2. The inflow openings 3 may furthermore have an orientation in the same direction, to get an even more directed inflow of the fluid. Fluid flow over the leaf-like elements 17 in this manner helps to close them faster.

FIG. 12 shows a cross sectional view of an embodiment of the present invention cardiac assist device, showing a further embodiment of the inner balloon element 5. In this embodiment, the inner balloon volume Vb2 is smaller than the inner cup volume Vc1 during operation. During inflation, this will ensure that the inner balloon element 5 will not trap or even encapsulate any fluid between the inner balloon element 5 and inner surface of cup element 2. During deflation, there will be fluid present between the inner balloon element 5 and inner surface of cup element 2, preventing a ‘vacuum’ of the area between and possible sticking of the two surfaces.

In the exemplary embodiment shown, the inner balloon element 5 is positioned distally within the cup element 2, i.e. nearer to the end cap 19 than to the outflow element 4. This will prevent an increased outflow resistance created by the inner balloon element 5 itself.

In addition, or alternatively, the inner balloon element 5 may be provided with a conical shape. This causes a predefined flow of fluid towards the outflow element 4.

Using one or more of the features described above with reference to FIG. 12, or above, it is possible to inflate the inner balloon element 5 in two phases, e.g. using variable compliance of the inner balloon element 5 material, using a specific shape (e.g. conical), or a combination thereof.

In the above described embodiments, a tube element 6 is used in communication with the inner balloon element 5. Fluid (air or helium) may be used to inflate and deflate the inner balloon element 5. In order to obtain a high enough frequency and timing of the inflation/deflation actions, the tube element 6 may be provided with specific technical features. E.g. to keep the flow resistance sufficiently low an inner diameter of between 4 and 8 mm², e.g. 5.3 mm² and a length of between 100 and 150 cm (e.g. 110 cm) of the tube element 6 is advantageous. This may be combined with an optimized inflation area of the inner balloon element 5 of 80 mm². Furthermore the material of the tube element 6 may be chosen to obtain a sufficient high radial stiffness (allowing rapid flow of e.g. helium in both directions), and at the same time a sufficiently high longitudinal push/pull strength in order to allow placing, positioning and retrieving the cardiac assist device 1.

The present invention has been described above with reference to a number of exemplary embodiments as shown in the drawings. Modifications and alternative implementations of some parts or elements are possible, and are included in the scope of protection as defined in the appended claims.

1. A cardiac assist device, comprising:
a cup comprising:
a cup wall defining an inner cup volume;
one or more in-flow openings arranged in the cup wall to allow a first fluid to flow into the cup during operation; and
an outflow element connected in fluid communication with the cup wall and having an aperture for expelling the first fluid during operation;
an inner balloon comprising a balloon wall defining an inner balloon volume, the inner balloon being positioned inside the cup; and
a tube in fluid communication with the inner balloon configured to inflate the inner balloon during a pumping operational mode and deflate the inner balloon during a filling operational mode,
wherein the cup is configured to counteract an outward directed force generated by the inner balloon during the pumping operational mode.

2. The cardiac assist device according to claim 1, wherein during the pumping and filling operational modes, the cup has a substantially constant inner cup volume.

3. The cardiac assist device according to claim 1, wherein the cup comprises a skeleton structure integrated with the cup wall.

4. The cardiac assist device according to claim 3, wherein the skeleton structure has a wire mesh structure.

5. The cardiac assist device according to claim 1, wherein the cup wall comprises an inner layer, an outer layer, or both an inner layer and an outer layer.

6. The cardiac assist device according to claim 1, wherein the cup further comprises a trans-valve section in communication with the outflow element.

7. The cardiac assist device according to claim 1, wherein the outflow element comprises a collapsible tubular element.

8. The cardiac assist device according to claim 3, wherein the skeleton structure comprises shape-memory material.

9. The cardiac assist device according to claim 3, wherein the outflow element comprises an outflow skeleton structure which is formed from an extension of the skeleton structure.

10. The cardiac assist device according to claim 9, wherein the outflow element comprises a collapsible tubular element is at least partially fixed to the outflow skeleton structure.

11. The cardiac assist device according to claim 1, further comprising a control unit arranged to control fluid flow through the tube during operation.

12. (canceled)

13. The cardiac assist device according to claim 1, wherein the combination of the cup and the inner balloon is adjustable to a transport mode, in which the maximum diameter of the combination is less than 7 mm.

14. (canceled)

15. The cardiac assist device according to claim 1, wherein the tube is a multi-lumen catheter.

16-17. (canceled)

18. The cardiac assist device according to claim 1, wherein the inner balloon has a conical shape.

19. The cardiac assist device according to claim 1, wherein the inner balloon comprises a multi-stage balloon assembly.

20. The cardiac assist device according to claim 1, wherein the one or more inflow openings comprise one-way valves.

21-26. (canceled)

27. The cardiac assist device according to claim 11, wherein the control unit is arranged to inflate and deflate the inner balloon at a frequency of pumping.

28. The cardiac assist device according to claim 27, wherein the control unit is arranged to set the frequency of pumping synchronously to a sensed heartbeat.

29. The cardiac assist device according to claim 27, wherein the control unit is arranged to set the frequency of pumping substantially higher than a sensed heartbeat.

30. The cardiac assist device according to claim 29, wherein the control unit is arranged to set the frequency of pumping to at least 2 times higher than a sensed heartbeat.

31. The cardiac assist device according to claim 30, wherein the control unit is arranged to set the frequency of pumping to at least a factor of 2-10 higher than a sensed heartbeat.

32-35. (canceled)

36. The cardiac assist device according to claim 4, wherein the cup comprises one or more circumferential restraining elements.

37. The cardiac assist device according to claim 3, wherein a portion of the skeleton structure is configured to pass through an aortic valve when the cardiac assist device is placed in a left ventricle of a patient.

38. The cardiac assist device according to claim 28, wherein the control unit is further arranged to set the frequency of pumping to at least 2 times higher than the sensed heartbeat.
